SEQUENCE LISTING (17 sequences)

SEQ ID NO: 1 - protein, 303 aa, Arabidopsis thaliana
MKLTYQFFIFWFFLPFFAISGDDDYKNLIFKGCANQKSPDPTGVFSQNLKNLFTSLVSQSSQSSFASVTSGTDNTTAVIGVFQCRGDLQNAQCYDCVSKIPKLVSKLCGGGRDDGNVVAARVHLAGCYIRYESSGFRQTSGTEMLFRVCGKKDSNDPGFVGKRETAFGMAENGVKTGSSGGGGGGGGFYAGQYESVYVLGQCEGSLGNSDCGECVKDGFEKAKSECGESNSGQVYLQKCFVSYSYYSHGVPNIEPLSGGEKRQHTERTIALAVGGVFVLGFVIVCLLVLRSAMKKKSNKYDAY

SEQ ID NO: 2 - DNA, 1213 bp, Arabidopsis thaliana
acatttttttgtctttaaacctcaaaacaaaagacaaaaaaaaaacggaaaactttcttttttccggacacataaaaacaacacaaactttattagacagaaacaaactagtaaaaagattttgaacatcaagaaacaaaaatctctctctgtctttctcaattatgaaactcacctatcaattcttcatcttctggttctttttaccattctttgcaatctccggtgatgatgattataaaaatctgatctttaaaggttgtgcaaatcagaaatctccagacccaactggtgttttctctcagaatctcaaaaacttattcacttctttagtttctcaatcatcacaaagctctttcgcttccgtaacctccggaactgataacaccaccgccgtgatcggtgtttttcagtgccgtggcgatctccaaaacgctcagtgttacgattgcgtctccaaaatccctaaactcgtttctaaactctgcggtggcggcagagatgacggtaatgtggtggcggctcgtgttcacctcgctggatgttatatccggtatgagagttcaggattccggcaaacttccggtacggagatgttgttccgtgtctgtgggaaaaaagattctaacgatcctgggtttgtcgggaaaagggagacggcgtttggtatggcggagaacggcgtgaaaactggatcatccggtggtggtggtggcggaggaggtttttacgcggggcagtatgagtcggtgtatgtgttgggacagtgtgagggtagtttgggaaattcagattgtggggaatgtgtgaaagatggatttgagaaagcaaagagtgagtgtggagagtcgaactcgggacaagtttaccttcagaagtgcttcgttagctatagttactactctcatggtgttcccaacatagagccattatcaggtggagagaagagacaacacacagaaaggacgatagctttggcggtgggaggagtttttgttttagggtttgtgattgtttgtttgttggttttgaggtctgccatgaagaagaagagtaataaatatgatgcttattgattcttatttttacctaccaattcttattttcccaattaaaaatttaacaaggtatattagaaaaatgattagtatagcatgtccaattgtatattcaattatgatattgatttaaatacaaataaattttgattttt

SEQ ID NO: 3 - protein, 201 aa, Arabidopsis thaliana
MAALVLSLLLLSLAGHSSASWCVCKTGLSDTVLQATLDYACGNGADCNPTKPKQSCFNPDNVRSHCNYAVNSFFQKKGQSPGSCNFDGTATPTNSDPSYTGCAFPTSASGSSGSTTVTPGTTNPKGSPTTTTLPGSGTNSPYSGNPTNGVFGGNSTGGTTGTGINPDYTTDSSAFALKNSSKLFICLLLIASSGFCSFLML

SEQ ID NO: 4 - DNA, 1025 bp, Arabidopsis thaliana
gagaagagacaagaagacacagaacaagtagccactctctcatctcaccacccctctttctctctacttgctctctatctctctctgtgtctctccactttctcttcttcttcttcttcttcttcttcttgctctagagatctctctatctccattcttttctctctctgtttctagaaagacaacagtgacattttgtctgaacgcatctatggctgctctggtgctttcacttctccttctatccttggctggacattctagtgcctcatggtgtgtgtgtaagacagggctgagtgatacagtgctacaggcaaccctagactatgcttgtggaaatggagcagattgtaatcctactaaaccaaaacaatcttgcttcaaccctgacaatgttaggtctcattgcaactatgcagtcaatagcttcttccaaaagaagggtcaatctcctggctcttgtaatttcgatggaactgccactcccactaactccgatcccagttatacaggttgtgcctttcctactagtgccagtggctctagcggcagcacaactgtgacacctggcacaaccaatccaaaaggcagcccaacgaccaccacacttcctggtagtggtaccaacagtccttattcagggaacccaaccaatggagtttttgggggaaatagcacaggaggcaccactgggacagggattaacccggattacacgacagacagcagcgcgtttgctctcaagaactcaagcaaattgttcatctgccttctcttgatcgcttcgagtggattctgctctttcctgatgctctaaggatgttaatggtctctggttctgtgggtgcacttagttatcgtttcgaggacattttggtctagtttagtggtaatctctgtctctgtgttatgggtctatgttatgtttttgtttgtaagaagatttcagctcttgcagttactcttaattacctctttgactgttctggtagatttgatgccagtgaaaaaaaatcttaatatat

SEQ ID NO: 5 - DNA, 20 bp, Artificial sequence
  Synthetic sequence Primer
ggccggcctggaggtggagg

SEQ ID NO: 6 - DNA, 21 bp, Artificial sequence
  Synthetic sequence Primer
ggccccagcggccgcagcagc

SEQ ID NO: 7 - DNA, 40 bp, Artificial sequence
  Synthetic sequence Primer
aaaaagcaggcttccgaatcatggctgctctggtgctttc

SEQ ID NO: 8 - DNA, 49 bp, Artificial sequence
  Synthetic sequence Primer
cacagctccacctccacctccaggccggccggcactagaatgtccagcc

SEQ ID NO: 9 - DNA, 50 bp, Artificial sequence
  Synthetic sequence Primer
tgctggtgctgctgcggccgctggggcctcatggtgtgtgtgtaagacag

SEQ ID NO: 10 - DNA, 35 bp, Artificial sequence
  Synthetic sequence Primer
agaaagctgggtcttagaccatcaggaaagagcag

SEQ ID NO: 11 - DNA, 24 bp, Artificial sequence
  Synthetic sequence Primer
acaagtttgtacaaaaaagcaggc

SEQ ID NO: 12 - DNA, 24 bp, Artificial sequence
  Synthetic sequence Primer
accactttgtacaagaaagctggg

SEQ ID NO: 13 - DNA, 33 bp, Artificial sequence
  Synthetic sequence Primer
cccccatggcttcatggtgtgtgtgtaagacag

SEQ ID NO: 14 - DNA, 31 bp, Artificial sequence
  Synthetic sequence Primer
cccgaattcttagtctgtcgtgtaatccggg

SEQ ID NO: 15 - protein, 35 aa, Arabidopsis thaliana
IALAVGGVFVLGFVIVCLLVLRSAMKKKSNKYDAY

SEQ ID NO: 16 - protein, 21 aa, Arabidopsis thaliana
IALAVGGVFVLGFVIVCLLVL

SEQ ID NO: 17 - protein, 18 aa, Arabidopsis thaliana
IALAVGGVFVLGFVIVCL

(19) United States
(12) Patent Application Publication
(10) Pub. No.: US 2009/0209002 A1
(43) Pub. Date: Aug. 20, 2009

(54) COMPOSITIONS AND METHODS RELATING TO CELLULAR TARGETING

(76) Inventors: Andrew Maule, Norfolk (GB); Carole Thomas, Norfolk (GB); Clare Simpson, Norfolk (GB)

Correspondence Address: MICHAEL BEST & FRIEDRICH LLP, ONE SOUTH PINCKNEY STREET, P O BOX 1806, MADISON, WI, 53701, US

(21) Appl. No.: 12/307,059

(22) PCT Filed: Jul. 2, 2007
(86) PCT No.: PCT/GB2007/002466

§ 371 Date: Dec. 30, 2008

(30) Foreign Application Priority Data
Jun. 30, 2006 (GB) 0613100.7

(51) Int. Cl. C12P 21/02; C07K 2/00; C07K 14/415; C12N 15/63; A01H 1/00; A01H 5/00; C12N 5/04

(52) U.S. Cl. 435/69.1; 530/300; 530/350; 530/402; 435/320.1; 800/278; 800/298; 435/410

(57) ABSTRACT

Plasmodesmal resident components are identified, characterized and isolated. Compositions comprising these components are described and methods of use thereof for plasmodesmal flux modulation and targeting are enabled. In a first embodiment, a novel plasmodesmal receptor-like protein, referred to herein as pldp1, reveals signals sufficient for targeting to plasmodesmata via the secretory pathway. In the second embodiment a novel plasmodesmal protein that is anchor into the external face of the plasma membrane and binds to callose is provided.

### FIELD OF THE INVENTION

Plasmodesmal compositions and methods provide control of trans-plasmodesmal flux and methods and means for targeting of molecules to plant Plasmodesmata.

### BACKGROUND OF THE INVENTION

Plasmodesmata are channels that cross the cell wall and establish symplastic continuity throughout most of the plant. Their importance has become apparent through the identification of a range of diverse non-cell autonomous functions dependent upon macromolecular communication. Hence, a range of transcription factors in the shoot apical meristem and at the root tip have functional roles at sites beyond the location of their production (1, 2). Similarly, some small RNAs generated as part of the RNA silencing process can act non-cell autonomously (3). Lastly, plant virus pathogens, which are restricted to the symplast, must utilise plasmodesmata to invade neighbouring cells (4). All of these macromolecules are above the experimentally defined size exclusion limits for plasmodesmata and point to highly regulated processes for their transport between cells. This is exemplified by plant viruses all of which encode so-called movement proteins (MPs) that interact and modify the properties of these structures to allow the passage of virus particles or other forms of ribonucleoprotein complexes (4). From the identification of proteins interacting with viral MPs, co-immunolocalisation studies applied to candidate proteins, and proteomics approaches, a number of plasmodesmata-associated proteins have been identified (5). However, despite the crucial roles shown by plasmodesmata for growth and development, plant defence, and pathogenesis almost nothing is known about the biogenesis, functional processes and constituent components of plasmodesmata. The identification of the constituent components of plasmodesmata has remained an outstanding, challenge, in plant biology and has hindered a fuller understanding of the non-cell autonomous control of plant development and the processes of tissue invasion by virus pathogens.

A fundamental question relating to the formation and function of plasmodesmata, is how proteins are recruited to this unique subcellular environment. Previous studies have indicated the importance of components of the cytoskeleton (6-10) and the post-Golgi vesicle trafficking systems (11) although in no case have the specific molecular addresses for delivery to plasmodesmata been identified.

Several lines of evidence suggest that cell to cell communication is acutely sensitive to callose deposition and resorbtion. Thus, it is known that plasmodesmata have a raised collar-like region at either end of the channel which is associated with callose (12), and inhibition of callose synthesis with 2-deoxy-D-glucose is known to prevent collar formation (13). Stress-related stimuli (plasmolysis; mild-cellulase digestion) are known to lead to callose deposition at plasmodesmata (14). Even rapid glutaraldehyde fixation has been found to result in callose deposition and neck constriction when viewed under EM (13, 15). Accordingly, it is known that this is an extremely rapid process. It is further known that callose plugs can block plasmodesmal transport. In the formation of cotton fibres the timing of the blockage of plasmodesmata regulates fibre length (16), and callose plugs have been proposed to be responsible for the maintenance of dormancy by symplastic isolation of the meristem (17). Further, in response to viral infection, antisense-tobacco deficient for β1-3-glucanase, which breaks down callose, showed an increase in callose deposition at plasmodesmata during virus infection and had a reduced size exclusion limit. These plants also showed reduced movement of Tobacco mosaic virus, Potato virus X and Cucumber mosaic virus (18). Also, expression of β1-3 glucanase from a virus vector increased susceptibility to that virus (19). In addition, transgenic expression of the TSWV NSm protein (MP) led to stunted growth and carbohydrate accumulation that was attributable to the blockage of plasmodesmata by callose (20) and finally, root growth inhibition associated with aluminium toxicity was associated with reduced cell-to-cell communication and callose deposition, reversal of which with 2-deoxy-D-glucose restored cell-to-cell communication (21).

Recently, a beta 1,3 glucanase has been reported to target to plasmodesmata (Levy et al 2007, Plant J. 49:669-682).

Nevertheless, there remains a need for a compositions and methods to provide both a better understanding of and methods for molecular targeting to plasmodesmata and modulating plasmodesmal flux. The present invention disclosure, in one embodiment, provides the use of Arabidopsis thaliana suspension cultures as a source of membrane proteins located in the cell wall, leading to the identification of plasmodesmal resident proteins, and the investigation of the path and principles by which their specific subcellular targeting is achieved, in turn leading to novel processes for regulating the flux of specific molecules that could influence growth and development, and defence. In another embodiment, this invention disclosure provides compositions, methods and means for controlling the influence of callose deposition on plasmodesmal flux by controlling the size and stability of the plasmodesmal aperture.

### SUMMARY OF THE INVENTION

Plasmodesmata provide the symplastic conduits for cell-to-cell communication throughout plant tissues. Despite their central role in growth and development and defence, resolving their modus operandii has remained a major challenge in plant biology. In a first embodiment of the present invention, we identify a novel family of membrane receptor-like proteins. The determinants for targeting to plasmodesmata were delimited to the C-terminal region of the transmembrane domain (TMD). By fusing the N-terminal secretory peptide and the C-terminal TMD to yellow fluorescent protein (YFP) we established that the TMD was sufficient to target proteins to these important structures. In a second embodiment, a novel callose-binding protein positioned to anchor plasmodesmata to the cell wall is identified, characterized and isolated.

### BRIEF DESCRIPTION OF THE DRAWINGS

FIG. 1: Pdlp1 targeting to plasmodesmata. Diagrams of a typical plasmodesma (PD) are shown in panel A (obtained from http://www.csse.monash.edu.au/˜cbetts/pd/pdpics.html). Plasmodesmata are plasma membrane lined channels that traverse the cell where there are callose deposits (arrow) at the outer limits that believed to regulate flux through the channel. The central area of the channel is occupied by an appressed form of the endoplasmic reticulum called the desmotubule. Transient expression of Pdlp1.GFP in a Nicotiana benthamiana leaf is shown in panels B-D. Optical confocal microscopy sections taken through the epidermis show the targeting of Pdlp1 to PD as punctate spots along adjoining cell walls (B). The top two panels of panel B show Pdlp1 labelling in the cell wall (left hand image), and the same image overlaid on an optical section of the corresponding transmitted light image (right hand image). The bottom two panels show targeting of Pdlp1 to the PD at the trichome-epidermis interface. Panels C and D show diagrammatic representations of observations made relating to the localisation of Pdlp1 after plasmolysis (C) and the colocalisation with callose (D). Upon plasmolysis Pdlp1 remained at punctuate structures within the cell wall. To confirm plasmolysis had occurred the plasma membrane was stained with FM4-64 and optical sections of Pdlp1 expression and FM4-64 labelling were overlaid. Spots of Pdlp1.GFP fluorescence were localised on the wall between the retracted plasma membranes of two adjacent cells. Callose containing PD were labelled with aniline blue fluorochrome. The same cell was monitored for Pdlp1 expression and showed co-localisation of Pdlp1 expression with the aniline blue fluorochrome.

FIG. 2: Organisation and Phylogenetic analysis of Pdlp1 and its homologues The amino acid domain structure of Pdlp1 is shown (A). The black box represents the signal peptide, as predicted by SignalP 3.0, and the grey box the transmembrane domain, as predicted by TMHMM 2.0. The transmembrane domain is followed by a short cytoplasmic tail. The DUF26 domain (Pfam PF01657) is shown in dark grey. Protein homology searches using the Pdlp1 amino acid sequence established the phylogenetic relationships and revealed the presence of two closely related families (B). Six of these genes (*) have been cloned and their subcellular location determined.

FIG. 3: Mutation analysis of the transmembrane domain and cytoplasmic tail of Pdlp1. Deletions were made in the transmembrane domain (TMD) and cytoplasmic tail (CT) of Pdlp1 (A). Forty eight hours after transient expression, in Nicotiana benthamiana, Pdlp1 deleted for the CT became localised as punctate structures in the cell wall (B). In contrast transient expression of Pdlp1 deleted of the CT and LVL from the TMD, abolished punctate labelling in the cell wall and resulted in targeting to the endoplasmic reticulum (C the right hand panel is an enlargement of a region (white box) within the left hand panel).

FIG. 4: The TMD of Pdlp1 is sufficient to target foreign proteins to plasmodesmata. A chimeric protein consisting of the signal peptide from Pdlp1, followed by the coding sequence for YFP (also called citrine) fused to the TMD and CT of Pdlp1 was constructed (A) and expressed transgenically in Arabidopsis. Upon plasmolysis of spongy mesophyll cells the chimeric protein remained in the cell wall at the areas of cell-cell contact (B and C) interpreted as PDs. Overlays of optical sections clearly show labelling at the cell to cell contact points (arrows). Background chloroplast autofluorescence is visible in panels B and C.

FIG. 5: Expression; of Pdlp2 in Arabidopsis thaliana. Transformed A. thaliana expressing Pdlp2.YFP were analysed by confocal microscopy. The protein was located at puncta on cell-wall interfaces. Examples shown are for leaf epidermal cells (A), leaf spongy mesophyll cells (B), and root epidermal cells (C and D; C is at a higher magnification). In each case the left panel shows the confocal fluorescence image and the right panel, the same image overlayed on the transmitted light image.

FIG. 6: Co-localisation of Pdlp2 with the callose marker alanine blue. Diagram to illustrate the observed location at cell-wall puncta for Pdlp2.YFP with respect to callose, as revealed by aniline blue staining. A=Pdlp2:YFP, B=callose, C=colocalisation.

FIG. 7: Phylogenetic analysis of Pdlp2 and subcellular targeting of family members. Phylogenetic analysis (A) showed Pldp2 (At5g61130) to be closely related to two other proteins, Pdlp2a and Pdlp2b (At5g08000 and At1 g18650, respectively). All three proteins are of similar size (about 190 amino acids) and possess a N-terminal signal sequence, a Glycosyl Phophotidylnositol (GPI)-anchor domain and an X8 domain. Transient expression of Pdlp2a.YFP in N. benthamiana (B) and transgenic expression of Pdlp2b.YFP in Arabidopsis thaliana (C) show that both related proteins are also targeted to plasmodesmata (fluorescent puncta on adjoining cell walls).

FIG. 8: Gel retardation assays for Pdlp2 binding to polysaccharides. Non-denaturing polyacrylamide gels without (no sugar) or with (sugar) added polysaccharide were used to assess the relative migration of: Pdlp2 fused to theoridoxin (P2), thioredoxin (Th) alone, BSA (negative control) or a sample of Ole 10 as a positive control (22; kindly provided by Dr R Rodriguez). In the presence of laminarin (callose/β1-3 glucan) (A) and to a lesser extent laminarihexose (6×β1-3 glucan) (B), Pdlp2 was retarded relative to thioredoxin alone (dotted line); Ole 10 was retarded only by laminarin (A and 22). Pdlp2 showed no retardation in the presence of CM cellulose (1-3 glucan) (C) or lichenan (1-3/1-4 mixed glucan) (D). For direct comparison, gels were run in sets of two, with and without a polysaccharide.

FIG. 9a: Pdlp1 Expression in Other Species

Optical sections through the lower epidermis of transgenic Arabidopsis expressing Pdlp1 (A, left). Transient expression of Pdlp1 in onion monolayers revealed punctate spots at the cell wall interface between adjacent cells (B, right).

FIG. 9b: Native Expression of Pdlp1 in Arabidopsis

Transgenic expression of Pdlp1 from its own promoter revealed the same punctate labelling seen following transient, expression (A). Plasmolysis leaves Pdlp1 punctate labelling in the cell wall (B arrows).

FIG. 10: Subcellular Targeting of Pdlp1 Homologues.

Optical sections through the spongy mesophyll of transgenic Arabidopsis expressing Pdlp homologues shows punctate labelling at the areas of cell-to-cell contact (some are marked with arrows). At3g60720 (A), At2g01660 (B), At1g04520 (C), At2g33330 (D) and At3g04370 (E).

FIG. 11: Expression profile of Pdlps using Genevestigator Meta-Analyser Depiction of organ and stages of growth (A) used in the analysis of Arabidopsis gene expression (B). The expression profiles of all 8 Pdlps are presented as a heat map relative to plant organs (B). All gene-level profiles were normalized for colouring such that for each gene the highest signal intensity obtains value 100% (dark) and absence of signal obtains value 0% (white).

FIG. 12: Alanine scanning mutagenesis of amino acids LVL within the transmembrane domain. Amino acids LVL at the end of the TMD of Pdlp1 lacking the CT were mutated singly or in combination and their effect on targeting determined after transient expression in Nicotiana benthamiana. From left to right the panels have the following mutations in the LVL sequence: LVL, LVA, LAL, AVL, LAA, AVA, AAL. Only those mutants that had the valine changed to an alanine no longer targeted PD, remaining in the endoplasmic reticulum.

Sequence annex: for ease of reference, this provides the sequences of Arabidopsis thaliana (previously) unknown protein (AT5G43980) mRNA and complete cds, and Arabidopsis thaliana (previously) unknown protein (AT5G61130) mRNA and complete cds. Other accessions referred to herein can readily be obtained online from publicly available sources using the appropriate accession no.

### DETAILED DESCRIPTION OF THE PREFERRED EMBODIMENTS OF THE INVENTION

In a first embodiment according to this invention, we have identified a first membrane-associated Plasmodesmata-located protein, referred to herein as Pdlp1. We have found that this protein targets to Plasmodesmata in diverse plant species. See Example 12. We have characterized this protein and found that Pdlp1 TMD contains a key determinant necessary and sufficient for plasmodesmal targeting. See Examples 13 and 14.

From these experiments, we conclude that the Pdlp1 family of membrane proteins are targeted to plasmodesmata. These proteins have related TMDs from which a consensus targeting signal is determined. The only other protein (excluding components of the cytoskeleton and resident ER proteins; 6) known to be stably incorporated in plasmodesmata is the 41 kDa reversibly glycosylated polypeptide (AtRGP2). AtRGP2 lacks a TMD and is proposed to associate with the cytosolic side of the Golgi membrane and therefore to reside within the cytosolic environment in plasmodesmata (23). The function of the RGPs remains unknown. The transportation of Pdlp1 is absolutely dependent upon the amino acids towards the C-terminus of the TMD and independent of the adjacent C-terminal tail.

The Pdlp1 proteins are exceptional in having a subcellular address specified by the TMD alone. It seems unlikely that this address is recognised while residing within the membrane. However, the location of the key determining amino acid between TMD amino acid 18 and 21 could result in it being exposed during its transition through the ER but incorporated within the plasma membrane at its final destination.

Pdlp1 is related to a larger family of proteins that share the 2×DUF26 domain configuration in common (24). Several of these genes have features of receptor-like kinases that are induced in response to treatments with pathogens or signalling molecules associated with pathogen attack (e.g. reactive oxygen species and salicylic acid; 25, 26, 27). These proteins have DUF26 domains located extracellularly and signal through a TMD to the cytoplasmic kinase (26). Logically, this places the C-terminus of Pdlp1 in the cytoplasm and the 2×DUF26 domains within the ER lumen and subsequently on the extracellular side of the PM, although this has not been verified experimentally. Presumably, Pdlp1 corresponds to a related family of receptors although the nature of the stimuli and the downstream signalling events are as yet unknown.

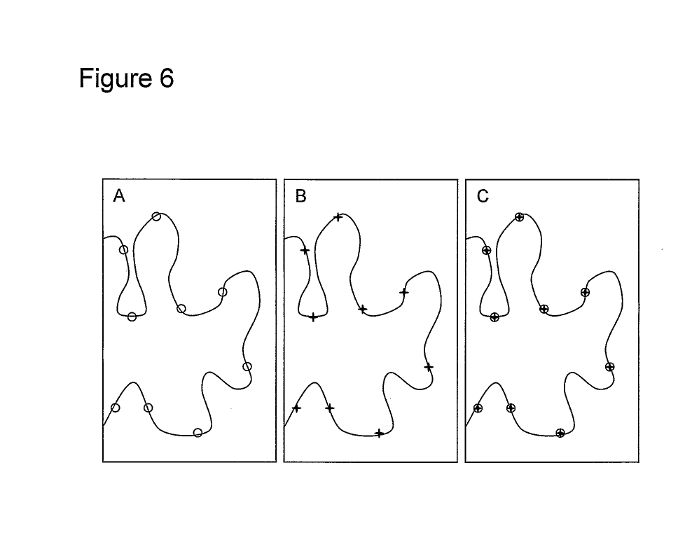

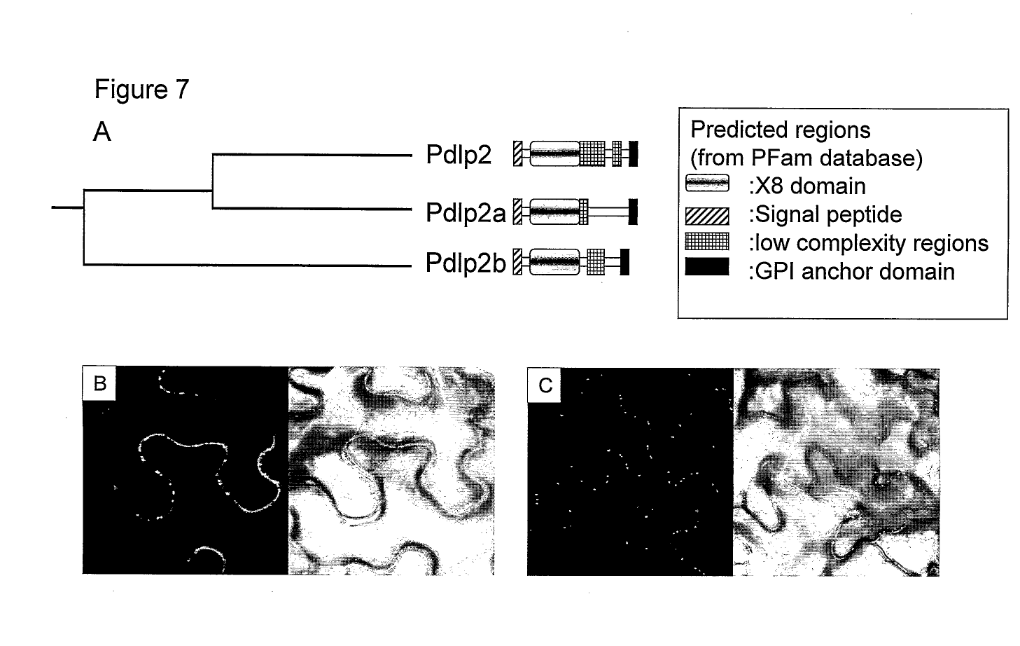

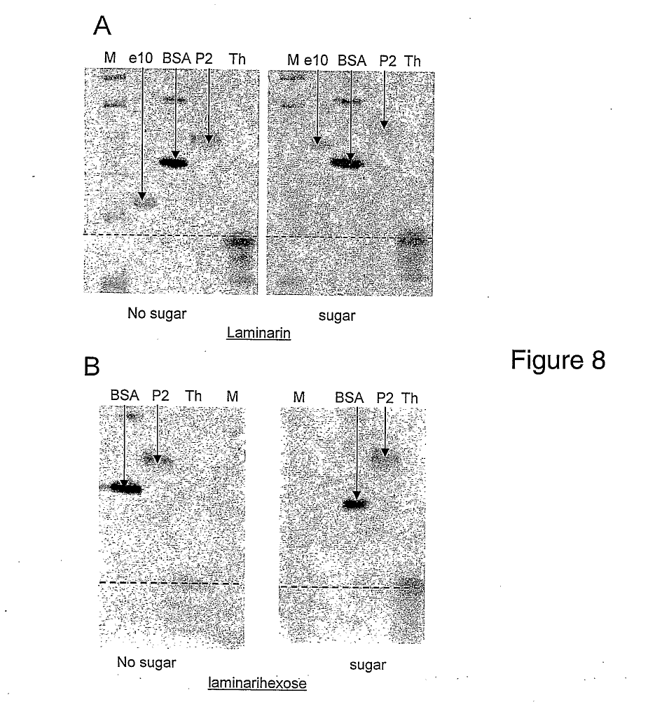

In a second embodiment, from a survey of GPI anchor proteins as GFP fusions we have identified a GPI protein as a novel Pd protein, which we refer to herein as Pdlp2. We show that this protein is targeted to Plasmodesmata in variety of systems, see Example 6 and 7 and FIGS. 5 and 7. We confirmed that these were Pds by plasmolysis and colocalisation with callose, see Example 10 and FIG. 6. On conducting phylogenetic analysis, we have shown that Pdlp2 is a member of a family of GPI proteins with a single X8 domain and other members of this family also target to plasmodesmata. X8 domains are a common feature of cell wall carbohydrate processing enzymes, and they are ancillary domains to the catalytic domain (28). X8 identified as a callose-binding protein in olive (22)

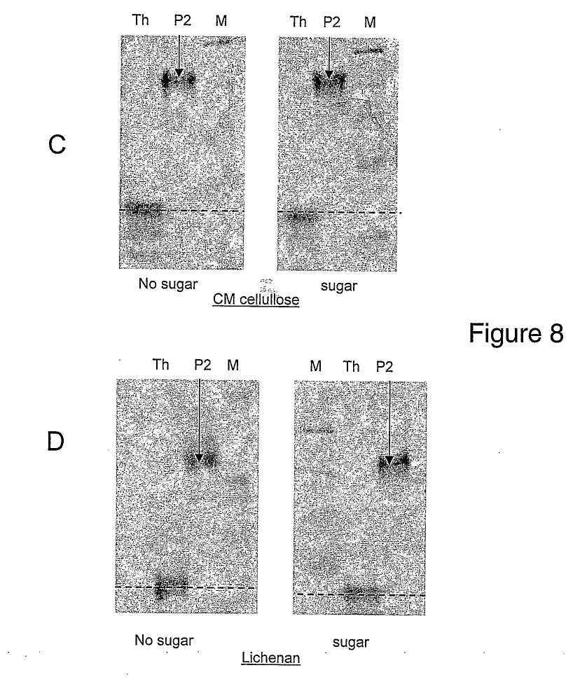

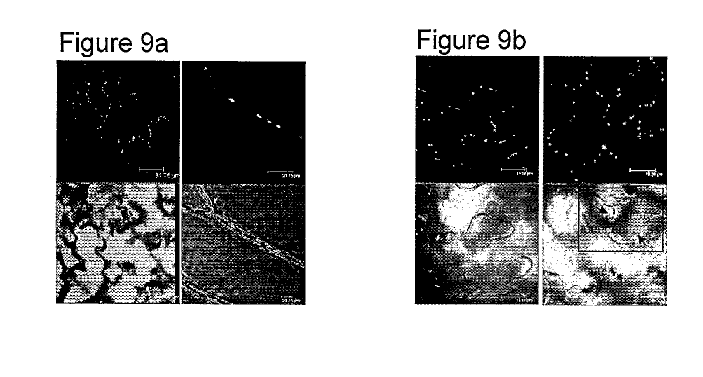

Genevestigator was used to analyse the expression profile of the gene (https://www.genevestigator.ethz.ch/). From this analysis, we found that Pdlp2 seems to be expressed throughout the plant at a low level. However, expression increases dramatically in the floral shoot meristem. Bacterial infection and general wounding results in a decrease in expression of Pdlp2 in plant tissues, which suggests closure of plasmodesmata as a plant stress response. We have expressed in and purified Pdlp2 from E. coli and subjected the purified enzyme to gel retardation assays in gels containing β1-3 glucan or other complex carbohydrates, and shown that it binds specifically to β1-3 glucan (callose), see Example 13 and FIG. 8.

From these experiments, we conclude that Pdlp2 is a novel plasmodesmal protein. As a GPI protein it is anticipated that it is covalently anchored on the external face of the plasma membrane. To achieve this destination Pdlp2 has specific signals for targeting to this location that distinguish it from GPI proteins incorporated into other areas of the plasma membrane. Callose deposition occurs in the cell wall at the neck region with the potential to constrict the plasmodesmal aperture. We demonstrate here that Pdlp2 binds to callose, and propose that Pdlp2 anchor plasmodesmata at the neck region. Understanding the regulation of Pdlp2 provides insights into the regulation of the size exclusion limit of Plasmodesmata and the process of plasmodesmal blockage that is integral to important aspects of plant development. It further provides an additional composition and methods and means for targeting expressed proteins linked to Pdlp2 or fragments thereof to the extracellular plasmodesmal address. Finally, by modification of Pdlp2, either by directly modifying the protein, by regulating its expression or by controlling targeting of the protein in the plant, plasmodesmal flux, via modulation of the interaction with callose or influencing accumulation, is enabled.

From these experiments and results, those skilled in the art will appreciate that we have identified, isolated and characterized Plasmodesmata-associated proteins. We have identified portions of these proteins which enable targeting of other proteins associated with these proteins or portions thereof (e.g. the TMD domain of Pdlp1) to either the external or internal environment of the plasmodesmal milieu. In so, doing, we have enabled multiple novel approaches to the control of the molecular components and constitution of plant Plasmodesmata and molecular flux therethrough.

Various further aspects and embodiments of the invention will now be described:

### Isolated Proteins and Equivalents

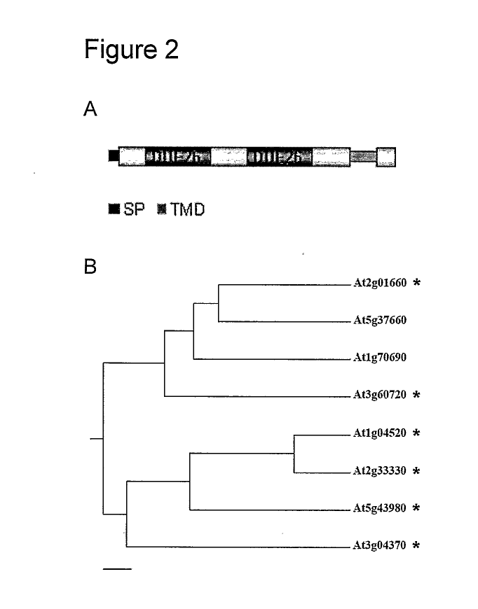

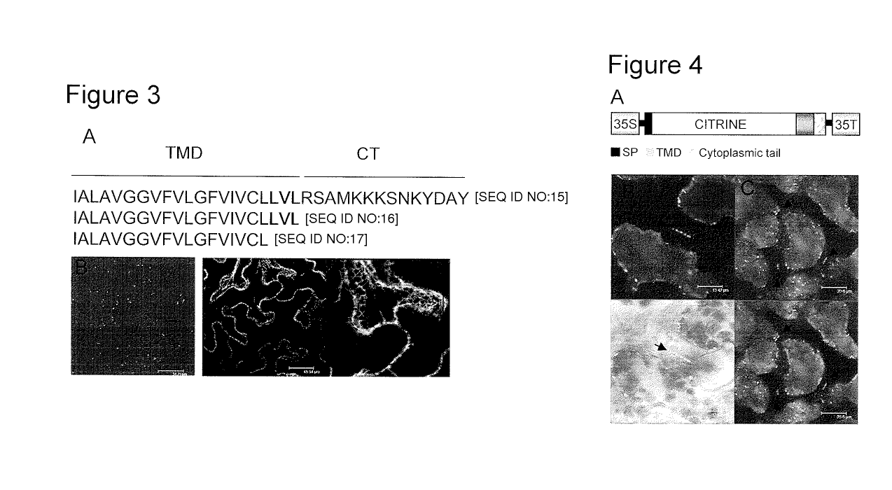

In one aspect there is provided an isolated plasmodesmata-associated protein, for example which may be selected from the group consisting of Pdlp1 and Pdlp2 and equivalents thereof (e.g. as encoded by any of the accessions given in FIG. 2B or described in FIG. 7 legend).

The sequence Annex appended hereto gives the mRNA and cds of At5g61130 (Pdlp1) and At5g43980 (Pdlp2) respectively. Other sequence accessions are also publicly available, albeit that they had not been characterised prior to the present invention.

Thus polypeptides according to the present invention may be provided isolated and/or purified from their natural environment, in substantially pure or homogeneous form, or free or substantially free of other polypeptides. Where used herein, the term “isolated” encompasses all of these possibilities. As discussed below, polypeptides may also be recombinant, and optionally present in a heterologous cell.

As used herein “equivalent” means a variant polypeptide (or nucleic acid) which shares homology with, or is identical to, all or part of the sequences discussed above.

Such variants may be inter alia used to target proteins to a plasmodesmal space, which can be assayed using the methods described herein e.g. with reference to Examples 12-13.

Although the following discussion is with respect to nucleic acids, it applies mutatis mutantdis to the corresponding peptides. Generally speaking variants may be:

(i) Novel, naturally occurring, nucleic acids, isolatable or identifiable using the sequences of the present invention e.g. alleles of the accessions described herein (which will include polymorphisms or mutations at one or more bases) or pseudoalleles. FIGS. 2, 11B, and 7 described certain sequences in the Pdlp1 and Pdlp2 families. Also included are paralogues, isogenes, or other homologous genes belonging to the same family as the Pdlp1 and Pdlp2 genes, which share conserved regions and functions with them e.g. from other plant species.

(ii) Artificial nucleic acids, which can be prepared by the skilled person in the light of the present disclosure. Such derivatives may be prepared, for instance, by site directed or random mutagenesis, or by direct synthesis. Preferably the variant nucleic acid is generated either directly or indirectly (e.g. via one or more amplification or replication steps) from an original nucleic acid having all or part of one of the Pdlp1 and Pdlp2 family sequences described herein.

Particularly included are variants which comprise only a distinctive part or fragment (however produced) corresponding to a portion of the sequence provided. The fragments may encode particular functional parts of the polypeptide e.g. capable of targeting proteins to a plasmodesmal space.

Some of the aspects of the present invention relating to variants will now be discussed in more detail.

Homology (similarity or identity) may be assessed, for example, using the Genetics Computer Group (GCG; Madison, Wis.) Wisconsin package version 10.0 program, ‘GAP’ (Devereux et al., 1984, Nucl. Acids Res. 12: 387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps.

Homology may be at the nucleotide sequence and/or encoded amino acid sequence level. Preferably, the nucleic acid and/or amino acid sequence shares at least about 65%, or 70%, or 80% identity, most preferably at least about 90%, 95%, 96%, 97%, 98% or 99% identity.

Homology may be over the full-length of the relevant sequence shown herein, or may be over a part of it, preferably over a contiguous sequence of about or greater than about 20, 25, 30, 33, 40, 50, 67, 133, 167, 200 amino acids or codons.

In a further aspect of the invention there is disclosed a method of producing a derivative nucleic acid comprising the step of modifying any of the sequences disclosed above.

Changes may be desirable for a number of reasons. For instance they may introduce or remove restriction endonuclease sites or alter codon usage.

Alternatively changes to a sequence may produce a derivative by way of one or more of addition, insertion, deletion or substitution of one or more nucleotides in the nucleic acid, leading to the addition, insertion, deletion or substitution of one or more amino acids in the encoded polypeptide.

Such changes may modify sites which are required for post translation modification such as cleavage sites in the encoded polypeptide; motifs in the encoded polypeptide for phosphorylation, GPI etc.

Other desirable mutations may be random or site directed mutagenesis in order to alter the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. Examples herein describe critical residues (e.g. V, F) in the TMD.

Deletion mutants and fragments (e.g. particular domains), plus fusion proteins, are discussed in more detail below.

The term ‘variant’ or ‘equivalent’ nucleic acid as used herein encompasses all of these possibilities. When used in the context of polypeptides or proteins it indicates the encoded expression product of the variant nucleic acid. Where used herein “Pdlp” polypeptide or nucleic acid may refer to any such variant.

### Recombinant Proteins and Methods of Production

This protein may be provided in recombinant form, by expression from encoding nucleic acid.

Nucleic acid according to the present invention (e.g. the vectors described below) may include cDNA, RNA, and genomic DNA. Where a DNA sequence is specified, e.g. with reference to a figure, unless context requires otherwise the RNA equivalent, with U substituted for T where it occurs, is encompassed. The nucleic acid molecules may be wholly or partially synthetic. In particular they may be recombinant in that nucleic acid sequences which are not found together in nature (do not run contiguously) have been ligated or otherwise combined artificially.

In one aspect of the present invention, the Pdlp nucleic acid described above is in the form of a recombinant and preferably replicable vector.

“Vector” is defined to include, inter alia, any plasmid, cosmid, phage or Agrobacterium binary vector in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication).

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

Specifically included are shuttle vectors by which is meant a DNA vehicle capable, naturally or by design, of replication in two different host organisms, which may be selected from actinomycetes and related species, bacteria and eucaryotic (e.g. higher plant, mammalian, yeast or fungal cells).

A vector including nucleic acid according to the present invention need not include a promoter or other regulatory sequence, particularly if the vector is to be used to introduce the nucleic acid into cells for recombination into the genome.

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell

By “promoter” is meant a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter. DNA operably linked to a promoter is “under transcriptional initiation regulation” of the promoter.

Thus this aspect of the invention provides a gene construct, preferably a replicable vector, comprising a promoter (optionally inducible) operably linked to a nucleotide sequence provided by the present invention, such as a Pdlp gene (or fragment thereof).

Particularly of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148). Suitable vectors may include plant viral-derived vectors (see e.g. EP-A-194809).

Suitable promoters which operate in plants include the Cauliflower Mosaic Virus 35S (CaMV 35S). Other examples are disclosed at pg 120 of Lindsey & Jones (1989) “Plant Biotechnology in Agriculture” Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

In a further aspect of the invention, there is disclosed a host cell containing a heterologous construct according to the present invention, especially a plant or a microbial cell.

The term “heterologous” is used broadly in this aspect to indicate that the gene/sequence of nucleotides in question (e.g. encoding a Pdlp polypeptide or part thereof) have been introduced into said cells of the plant or an ancestor thereof, using genetic engineering, i.e. by human intervention. A heterologous gene may replace an endogenous equivalent gene, i.e. one which normally performs the same or a similar function, or the inserted sequence may be additional to the endogenous gene or other sequence. Nucleic acid heterologous to a plant cell may be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

The host cell (e.g. plant cell) is preferably transformed by the construct, which is to say that the construct becomes established within the cell, altering one or more of the cell's characteristics and hence phenotype e.g. with respect to plasmodesmal structure or activity.

Thus a further aspect of the present invention provides a method of transforming a plant cell involving introduction of a construct as described above into a plant cell and causing or allowing recombination between the vector and the plant cell genome to introduce a nucleic acid according to the present invention into the genome.

Thus the invention provides, inter alia a recombinant vector (e.g. an expression vector) which comprises the nucleic acid encoding a polypeptide as described above e.g. Pdlp1, Pdlp2 or variants thereof (e.g. which shares at least about 50%, 60%, 70%, 80% or 90% identity with either), or fusions with components thereof (as described herein). The nucleic acid may optionally include all or part of the cDNA sequence shown in the Sequence annex, or be degeneratively equivalent thereto.

The invention further encompasses a host cell transformed with nucleic acid or a vector according to the present invention (e.g. comprising the Pdlp sequence) especially a plant or a microbial cell. In the transgenic plant cell (i.e. transgenic for the nucleic acid in question) the transgene may be on an extra-genomic vector or incorporated, preferably stably, into the genome. There may be more than one heterologous nucleotide sequence per haploid genome.

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

Thus the invention provides a method for producing a transgenic plant, which method comprises the steps of:

(a) introducing the vector described above into a host cell, and optionally causing or allowing recombination between the vector and the host cell genome such as to transform the host cell, wherein the host cell is a plant cell,
(b) regenerating a plant from the transformed plant cell.

Plants which include a plant cell according to the invention are also provided.

In addition to the regenerated plant, the present invention embraces all of the following: a clone of such a plant, seed, selfed or hybrid progeny and descendants (e.g. F1 and F2 descendants). The invention also provides a plant propagule from such plants, that is any part which may be used in reproduction or propagation, sexual or asexual, including cuttings, seed and so on. It also provides any part of these plants, which in all cases include the plant cell or heterologous Pdlp DNA described above.

### Modification of Expression Levels

Methods as described above may be used to alter the expression of Pdlp proteins, for example modifying naturally or artificially the expression levels or location of Pdlp2 or a homolog or ortholog thereof, may be used to effect changes in callose deposition.

The present invention also encompasses the expression product of any of the coding Pdlp nucleic acid sequences disclosed and methods of making the expression product by expression from encoding nucleic acid therefore under suitable conditions, which may be in suitable host cells.

In addition to use of the nucleic acids of the present invention for production of functional Pdlp polypeptides, the information disclosed herein may also be used to reduce the activity Pdlp activity in cells in which it is desired to do so.

For instance down-regulation of expression of a target gene may be achieved using anti-sense technology.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Rothstein et al, 1987; Smith et al, (1988) Nature 334, 724-726; Zhang et al., (1992) The Plant Cell 4, 1575-1588, English et al., (1996) The Plant Cell 8, 179-188. Antisense technology is also reviewed in Bourque, (1995), Plant Science 105, 125-149, and Flavell, (1994) PNAS USA 91, 3490-3496.

An alternative to anti-sense is to use a copy of all or part of the target gene inserted in sense, that is the same, orientation as the target gene, to achieve reduction in expression of the target gene by co-suppression. See, for example, van der Krol et al., (1990) The Plant Cell 2, 291-299; Napoli et al., (1990) The Plant Cell 2, 279-289; Zhang et al., (1992) The Plant Cell 4, 1575-1588, and U.S. Pat. No. 5,231,020. Further refinements of the gene silencing or co-suppression technology may be found in WO95/34668 (Biosource); Angell & Baulcombe (1997) The EMBO Journal 16, 12:3675-3684; and Voinnet & Baulcombe (1997) Nature 389: pg 553.

RNAi is widely used as a technique to suppress certain target genes, to create ‘knock-outs’, e.g. in functional genomic programs. More specifically this is done using hairpin constructs that are designed to trigger PTGS of the target gene, based on homology of sequences. Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi) (See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245). Thus siRNA duplexes containing between 20 and 25 bps, more preferably between 21 and 23 bps, of native Pdlp sequences form one aspect of the invention e.g. as produced synthetically, optionally in protected form to prevent degradation.

### Active Components of Proteins

Particular regions, domains or fragments (which are all synonymous with “components”) of Pdlp have utility in their own right, for example in the context of fusion polypeptides based on the plasmodesmal-targeting domains (e.g. TMD). Thus nucleic acids encoding these domains, or fusion proteins comprising them, form one embodiment of this aspect of the present invention.

Thus the present invention also provides for the production and use of fragments of the full-length polypeptides disclosed herein, especially active portions thereof. An “active portion” of a polypeptide means a peptide which is less than said full length polypeptide, but which retains an essential biological activity. In particular, the active portion retains the ability to target associated material (e.g. protein) to the plasmodesmal space.

A “fragment” of a polypeptide means a stretch of amino acid residues of at least about five, to seven contiguous amino acids, often at least about seven to nine contiguous amino acids, typically at least about nine to 13 contiguous amino acids and, most preferably, at least about 20 contiguous amino acids. Fragments are preferably derived from the TMD of a Pdlp, and preferably include at least the critical V (or F) described herein.

Use of recombinant Pdlp protein, or a fragment (e.g the domains discussed above) thereof, to target a protein linked thereto to the plasmodesmal space, forms one aspect of the invention.

The invention further provides an isolated component of a plasmodesmata-associated protein which is sufficient to target a protein linked thereto to the plasmodesmal space.

The isolated component of a plasmodesmata-associated protein may optionally be the transmembrane domain (TMD) of Pdlp1. Or it may be the TMD of the equivalent proteins discussed above, or a mutant of any of these which retains targeting activity.

The isolated component of the plasmodesmata-associated protein may optionally be part of Pdlp2. This may be a component which specifies targeting to the extracellular domain of the plasma membrane for interaction with the cell wall, and may optionally bind callose.

### Fusion Proteins and Methods of Production

Fusion proteins of heterologous polypeptides with “components” of the invention may conveniently be prepared with vectors which permits the introduction of heterologous sequences in frame with all or part of the Pdlp sequence, as described in the Examples herein.

Thus the invention provides a chimeric or fusion polypeptide comprising a Pdlp domain as described herein fused to a polypeptide heterologous to said Pdlp domain. Also provided are corresponding encoding nucleic acids, vectors, plants, and methods of use as described herein.

### Further Methods and Uses

The invention further provides a method for targeting a protein to the plasmodesmal milieu which comprises linking said protein to an isolated plasmodesmata-associated protein or to a component of a plasmodesmata-associated or related protein which is sufficient to target said protein linked thereto to the exterior or interior of the plasmodesmal space, for example any of those discussed above.

Such methods may be employed, for example, in controlling flux through plant plasmodesmata by modifying the composition of the plasmodesmata (e.g. by targeting to the plasmodesmata proteins that physically obscure the flux channel).

One application of this might be in modifying the extent of post-transcriptional gene silencing in the plant, but controlling the passage of siRNAs through the plasmodesmata

Thus the invention provides a method which comprises targeting novel proteins to the plasmodesmata by targeting proteins of interest to said space by linking said proteins to an isolated plasmodesmata-associated protein or to a component of a plasmodesmata-associated protein (e.g. any discussed above) which is sufficient to target said protein linked thereto to the plasmodesmal space.

Such methods may comprise targeting novel proteins with a defined binding specificity to plasmodesmata to modify specific molecular flux through plasmodesmata e.g. such as to modify the effectiveness of callose accumulation as a means for controlling plasmodesmal flux.

This may also be achieved by modifying naturally or artificially the expression levels or location of Pdlp2 or a homolog or ortholog thereof, as discussed above.

Any of these methods may for example be used to modify plant development or plant defence responses.

In a further embodiment, antibodies raised to the Pdlp polypeptides or peptides can be used in the identification and/or isolation of variant polypeptides, and then their encoding genes, or in immunolocalisation. Methods of obtaining antibodies are well known to those in the art.

A further aspect of the invention is any of the primers described herein e.g. for use in preparing fusion proteins.

Thus in summary, applications for the technology include:

1) An isolated polypeptide which is (i) a plasmodesmata-associated protein, or (ii) an isolated component of a plasmodesmata-associated protein, which polypeptide is sufficient to target a protein linked thereto to the plasmodesmal space. Also methods in which these proteins are over- or under-expressed to modify plasmodesmata activity.

2) Methods and materials for controlling flux through plasmodesmata through controlling or changing the size of the aperture. In particular the technology disclosed herein may be used to modify the region in or around the “neck” of the plasmodesmata (i.e. the region by the opening on the external cell wall side) for example by introduction of novel proteins, modification of Pdlp2, or indirectly by modification of callose (beta 1,3-glucan) deposition in this area of the cell wall. As described herein, it is believed that Pdlp2 is covalently attached to the external face of the plasma membrane at its C-terminus and the N-terminus binds to callose in the wall.

3) Methods and materials for controlling flux through the plasmodesmata by way of introducing modified or novel polypeptides at or around the internal opening or within the plasmodemata. As shown in the Examples herein, the sequences of the invention (e.g. Pdlp1) may be used target sequences in this way. Proteins may be selected which are cleaved or degraded in response to developmental or environmental stages and thereby increasing flux at these times. Such modification of flux may be used to control plant phenotype—for example the length of cotton fibres.

4) Methods and materials for introducing novel functionalities or specificities into the aperture of the plasmodesmata, for example by fusion of enzymes or antibodies (e.g. a single chain antibody) to the targeting sequences described herein. This may be used, for example, to specifically modify the function of viral movement proteins, hence control viral spread.

Having generally described this invention, the following examples and experimental detail is provided to further enable this invention. However, those skilled in the art will appreciate that the specifics of these examples should not be read as limiting on the invention. Rather, for purposes of understanding the scope of the invention disclosed herein, those skilled in the art are referred to the claims appended hereto.

### EXAMPLES

### Example 1

### Gateway Cloning of Pdlp1

Gateway technology (Invitrogen) was used to generate all the clones in this disclosure. Pdlp gene specific sequences were amplified by PCR using Phusion DNA polymerase (NEB). Gene sequences were engineered to carry partial attB sites on either end. After purification, full length attB sites were reconstituted by an additional round of PCR using attB adaptor primers. The resulting DNA fragment was purified and transferred by recombination into the entry vector pDONR207 (Invitrogen) using BP clonase II (Invitrogen) following the manufacturers conditions. The sequence of the resulting pDONR clone was verified using BIG Dye 3. The Pdlp sequence was transferred by recombination to the indicated binary destination vector using LR clonase II (Invitrogen) following the manufacturers conditions.

### Example 2

### Cloning of Pdlp1 Family CDS

Primers were designed to the 5′ and 3′ ends of the appropriate coding sequences (CDS). The 5′ primer included a partial attB1 site, a Kozak sequence and the ATG of the CDS. The 3′ primer was missing the termination codon and included a partial attB2 site. The CDS was PCR amplified, using Phusion DNA polymerase (NEB) from a pool of cDNA made from Arabidopsis Columbia RNA. Gateway cloning was used to transfer the Pdlp CDS into the entry vector pDONR207 (Invitrogen). The Pdlp CDS in the resulting pDONR-Pdlp clone was transferred by recombination to the binary destination vector pB7FW2.0 (29). Agrobacterium GV3101 was transform via electroporation with the resulting binary clone. Transformed GV3101 was used for transient and transgenic expression of Pdlp CDSs.

### Example 3

### Cloning of Pdlp 1 Promoter Construct

Primers were designed to a region 1.5 Kb upstream of the ATG of Pdlp1 and to the 3′ end of Pdlp1. The 5′ primer contained a partial attB1 site, the 3′ primer was missing the termination codon and included 19 nt of eGFP sequence. The promoter plus CDS was amplified using Phusion DNA polymerase. eGFP was PCR amplified using primers to the 5′ end and a 3′primer carrying a stop codon plus a partial attB2 site. Both the eGFP and Pdlp1 DNA fragments were purified and joined by overlap PCR using attB adaptors to reconstitute the attB sites. Gateway technology was used to recombine the resulting 3.6 Kbp fragment into the entry vector pDONR207 (Invitrogen) and transfer it to the binary destination vector pEarleygate 301 (30). Agrobacterium GV3101 was transformed by electroporation with the resulting binary clone and was used for Arabidopsis transformation.

### Example 4

### Mutagenesis of Pdlp1

PCR mutagenesis of Pdlp1 was used to create deletions and alanine substitutions of the transmembrane domain and cytoplasmic tail. Primers were made that incorporated the required mutation. All cloning was carried out using Gateway technology, the entry vector pDONR207 and destination vector pB7FWG2.0.

### Example 5

### Cloning of pB7WG-SP-CIT-TMC

Primers were designed to the 5′ and 3′ end of the signal peptide of Pdlp1. The 5′ primer contained a partial attB1 site, a Kozak sequence and the ATG of the gene, the 3′ primer contained 30 nt of the 5′ end of YFP (also called ‘citrine’) coding sequence. A second set of primers were made to the 5′ end of the Pdlp1 transmembrane domain and the 3′ end of the Pdlp CDS. In addition to TMD sequence the 5′ primer contained 28 nt of 3′ citrine sequence, the 3′ primer contained a partial attB2 site. The coding sequence for citrine, Pdlp1 signal peptide and the Pdlp1 transmembrane domain were PCR amplified using Phusion DNA polymerase) (NEB). Overlap PCR of the purified DNA fragments was carried out using attB adaptor primers and the resulting 1 Kbp fragment cloned via Gateway technology into pB7WG2.0 (29).

### Example 6

### Arabidopsis Transformation

Flower dip transformation was carried out according to Clough and Bent (31). Transgenics seedlings were identified by spraying the germinated TO seed with ‘Challenge’ (herbicide) and expressing lines selected by screening the survivors using a confocal microscope.

### Example 7

### Transient Expression in Nicotiana benthaniana

Agrobacterium GV3101 containing the test Pdlp destination clone were grown overnight in LB plus the appropriate antibiotics. The bacteria were collected and resuspended in 3 ml of 10 mM MgCl₂ containing 100 μM acetosyringone. After a minimum of 2 h at room temperature the culture was diluted to an OD_{600 nm} of 0.2-0.5. Two leaves on a young plant at the 4-6 leaf stage were infiltrated with the Agrobacterium culture and left for 48-60 h before examining using the confocal microscope.

### Example 8

### Transient Expression in Onion Cell Monolayers

The epidermis was dissected from the onion and placed on 6% Agar MS plates. Biolistic bombardment using the BioRad gene gun delivered gold particles coated in pBFWG-Pdlp1. The onion epidermis was incubated in a growth room for 48-60 h before being examined using a confocal microscope.

### Example 9

### Plasmolysis of Plant Tissue

Plant tissue was plasmolysed using either 0.7M mannitol (when using FM4-64) or 30% glycerol. FM4-64 was dissolved in 0.7M mannitol at a concentration of 33 μM. In each case the solution was infiltrated into the tissue and the tissue monitored for plasmolysis.

### Example 10

### Staining of Callose with Aniline Blue Fluorochrome

Aniline blue fluorochrome was used at 0.1 mg/ml and infiltrated into the transformed plant tissue before being analysed by confocal microscopy.

### Example 11

### Confocal Microscopy

Transformed plant tissue was imaged using a Leica SP2 inverted confocal microscope (Milton Keynes, UK), and ×40 or ×60/1.32 oil objectives. FM4-64 and GFP were excited using 488 nm light from an Argon ion laser and the emitted light was captured at 630-680 nm and 505-555 nm respectively. Aniline blue fluorochrome was excited with a 405 nm laser (30% strength) and the emitted light captured between 460-500 nm.

### Example 12

### Plasmodesmata-Located Protein 1 (Pdlp1) Targets to Plasmodesmata in Diverse Plant Species

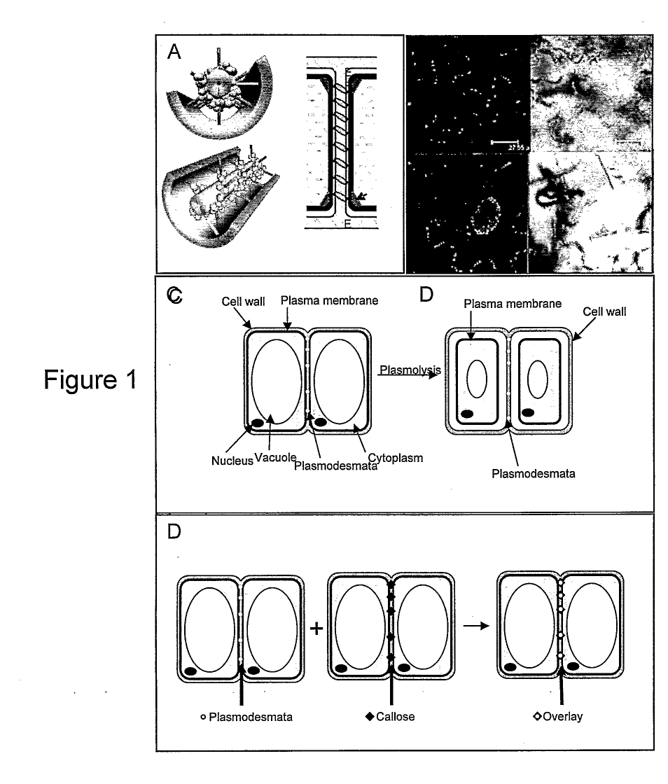

From a list of cell wall-associated membrane proteins derived from highly purified Arabidopsis cell walls (32), a C-terminal fusion of At5g43980 to GFP was constructed and expressed transiently in Nicotiana benthamiana leaves, Arabidopsis suspension cells and onion epidermal monolayers, and transgenically in Arabidopsis. In all cases, the fusion protein was targeted to punctate spots on the cell wall (FIG. 1B and FIG. S1), although extreme over-expression associated with transient, expression in N. benthamiana sometimes resulted in Pdlp1-GFP accumulation within the plasma membrane (FIG. S1). Confirmation that these sites were plasmodesmata was obtained by demonstrating co-localisation with callose (FIG. 1C). This punctate pattern was retained after plasmolysis. Particularly notable was that in spongy mesophyll cells the fusion protein was restricted to puncta along the areas of cell-to-cell contact and absent from the remainder of the cell wall (FIG. 1D). We refer to this protein as plasmodesmata-located protein 1 (Pdlp1).

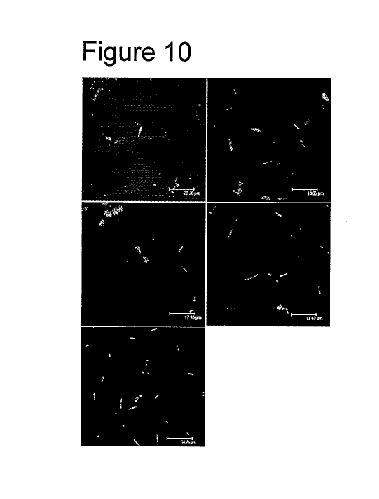

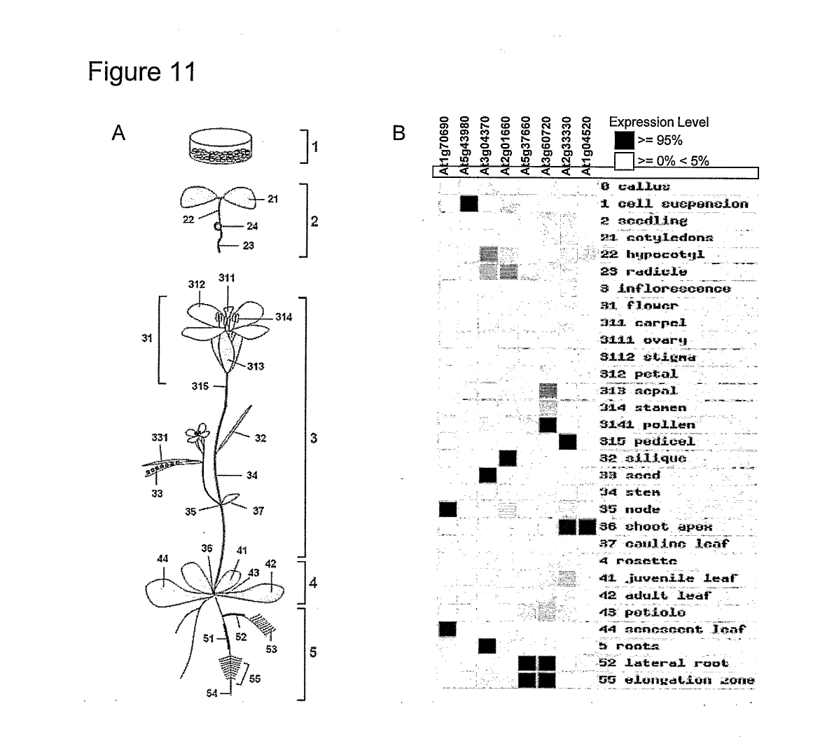

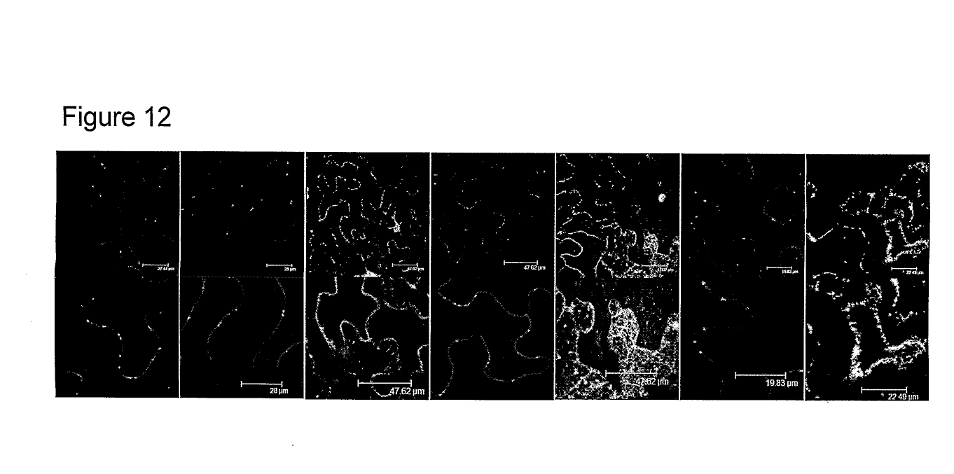

Sequence analysis showed Pdlp1 to have a domain structure conserved in a small family of plant-specific proteins (FIG. 2 and FIG. 10) including representatives from Arabidopsis, rice, and Phaseolus. Briefly, the proteins are predicted type I membrane proteins comprising an N-terminal signal peptide, a region containing 2×DUF26 (domains of unknown function) domains, a single transmembrane domain and a short C-terminal tail. Unfortunately, these domains give no direct clues as to protein function although DUF26 domains are also found in receptor-like kinases. DUF26 domains have a conserved C-X8-C-X2-C motif which is distinct from the Cys-rich regions found in S-locus glycoproteins (24). Arabidopsis genetic knockouts of the Pdlp family show no obvious phenotype, so pointing to potential redundancy within the family or highly specific and subtle roles in molecular trafficking (data not shown). Expression analysis based upon public data (33) showed that members of the family were expressed in all plant tissues (FIG. 11) although they appear to vary significantly in their relative abundance in different tissues. Notably, At5g43980 is highly expressed in suspension cells which may explain why it was detected in our proteomic screen (32).

### Example 13

### Pdlp1 TMD Contains a keV Determinant Necessary for Plasmodesmal Targeting

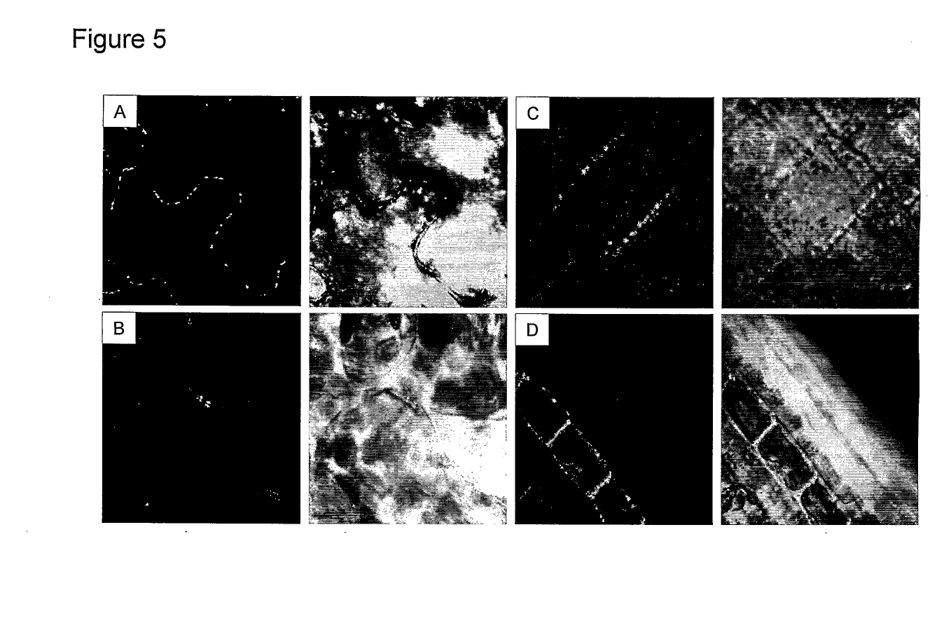

It has been proposed (34) that a TMD-length rule applies to proteins translocated from the ER to the plasma membrane (PM) via the Golgi to correspond with the increasing thickness of the membranes along the path. Hence, ER proteins may have TMDs of 17 or 18 amino acids but PM proteins require a TMD of 21 or 22 amino acids. Bioinformatic analysis of the Pdlp1 family identified a common TMD of 21 amino acids upstream of a short but variable length C-terminal tail. To assess the importance of the C-terminal tail and the TMD, two constructs were tested for At5g43980, a deletion of the C-terminal tail and a larger deletion of the same region but extended to include the three C-terminal amino acids of the TMD, shortening it to 18 amino acids (FIG. 4A). Transient and transgenic expression showed that the protein lacking the C-terminal tail was still targeted to plasmodesmata whereas the larger deletion lacking three amino acids-(LVL) of the TMD was retained in the ER (FIG. 4B, C). Clearly, the three C-terminal amino acids of the TMD must contain a key determinant for targeting. To assess which of these amino acids were important, alanine substitutions were made singly or in combination at the three positions (FIG. 4A). These alanine substitutions retained the predicted length of the TMD. In all cases, plasmodesmal targeting was retained unless the V to A substitution was included in the mutated protein, in which case the protein was retained in the ER (FIG. S5). This critical valine residue is not conserved across the Pdlp1 family; other members having a phenylalanine in the same position. We expect that similar mutagenesis for At2g33330 also shows a loss of plasmodesmal targeting after a F to A substitution in transgenic Arabidopsis.

### Example 14

### Pdlp1 TMD is Sufficient for Plasmodesmal Targeting

The finding that Pdlp1 achieved plasmodesmal targeting in the absence of the C-terminal tail led us to ask whether the TMD alone would be sufficient. To assess this two constructs were tested, yellow fluorescent protein (YFP; this has higher stability in the acidic extracellular environment; Tian et al 2004) was fused to the TMD plus C-terminal tail or to the TMD alone. Transgenic expression of the former constructs showed targeting of YFP to the plasmodesmata and this location was retained after plasmolysis (FIG. 5A, B). For the latter, based upon the behaviour of At5g43980 lacking the CT tail, we expect that a similar pattern of behaviour is revealed. For the former construct, YFP fluorescence was also visible at discrete locations in the cytoplasm. The nature of these sites is not known at present. Nevertheless, our experiments have uniquely identified a short TMD sequence with the capacity to target novel proteins to plasmodesmata.

### Example 15

### Cloning of Pdlp2 as a YFP Fusion Protein

The YFP gene was inserted into Pdlp2 between the N-terminal signal sequence and the rest of the protein, using nested PCR. Forward and reverse primers (5′ GGC CGG CCT GGA GGT GGA GG 3′ and 5′ GGCCCCAGCGGCCGCAGCAGC 3′) for YFP (also called pCitrine; Tian et al 2004) and two sets of Pdlp2 primers (P1/P2 and P3/P4) were used. P1 (5′ AAAAAGCAGGCT tccgaatcATGGCTGCTCTGGTGCTTTC 3′) and P2 (5′-CACAGCTCCACCTCCACCTCCAGGCCGGCCGGCACTAGAATGTCCAGCC-3′) amplified the N-terminal signal sequence. P1 contained sequences that partially overlap the Gateway primers (underlined area) and a Kozak translational consensus start site sequence (in lower case lettering) and P2 contained a region overlapping YFP, (underlined region). P3 (5′ TGCTGGTGCTGCTGCGGCCGCTGGGGCCTCATGG TGTGTGTGTAAGACAG 3′) and P4 (5′AGAAAGCTGGGTCTTAGACCATCAGGAAAGAGCAG 3′) amplified the rest of the gene. P3 contains a region overlapping YFP (underlined) and P4 contains a partially overlapping area with the Gateway primers (underlined). All fragments were amplified separately then nested together in two stages. Fragments were amplified from At5g61130 CDS and YFP using Ex taq (Takara Bio). A second PCR reaction using primers P1 and YFPrev fused the N-terminus of At5g61130 and YFP together. A third reaction used a pair of gene-nonspecific Gateway primers to fuse the N-YFP to the rest of the gene. The forward primer contains the attB1 sequence 5′-ACAAGTTTGTACAAAAAGCAGGC-3′ and the reverse primer the attB2 sequence 5′-ACCACTTTGTACAAGAAAGCTGGG-3′. The PCR product was recombined into the Gateway donor vector pPONR207 (Invitrogen), using BP Clonase mixture (Invitrogen) and transformed into the DH5α strain of E. coli.

YFP-tagged Pdlp2 was transferred from pDonor into its binary destination vector pB7WG0.2 and transformed into the DH5α strain of E. coli. The cloned products were verified by DNA sequencing.

### Example 16

### Pdlp2 Expression in Escherichia coli

At5g61130 (Pdlp2) CDS was cloned between the NcoI and EcoRI sites of pET32 (Invitrogen) after PCR amplication using FW (5′CCCCCATGGCTTCATGGTGTGTGTGTAAGACAG 3′) and RV (5′CCCGAATTCTTAGTCTGTCGTGTAATCCGGG 3′) primers. These primers included the NcoI and EcoRI restriction sites for cloning and a translational stop codon in the reverse primer. The clone was transformed into DH5α strain of E. coli and the insert verified by DNA sequencing. After verification the clone was further transformed into E. coli BL21 Des pLyse strain for protein expression. Cloning CDS into pET32 results in the expression of a fused protein with a 6×His tag, N-terminal to thioredoxin, in turn N-terminal to the inserted CDS, a strategy designed to increase the solubility of the expressed protein in E. coli. As a control, empty pET32 expressing thioredoxin alone was processed in parallel.

For protein expression and purification, bacterial cultures were grown at 37° C. to OD 0.7-1, then induced using 0.5 mM IPTG. At this point the temperature was reduced to 21° C. and the bacteria left to grow overnight. The resulting bacteria were collected by centrifugation, resuspended in buffer (50 mM sodium phosphate pH 8, 300 mM sodium chloride, 20 mM imidazole) and disrupted using a French press. After centrifugation at (30,000 g) the soluble fraction was retained.

The His-tagged proteins was purified from the soluble fraction using FPLC with a 1 ml HiTrap Chelating HP column (Amersham). The Pdlp2 containing fractions were further FPLC purified using an Amersham Hiload 16/60 Superdex 200 gel filtration column and the resulting fractions concentrated.

### Example 17

### Gel Retardation Experiments

Pdlp2-binding to complex carbohydrates was assessed using gel retardation assays as described by Barral et al (22). Briefly, non-denaturing PAGE gels (12% w/v poly acrylamide gels in 0.3M Tris buffer, pH 8.0) were prepared with 3-5 mg/ml of the different polysaccharides added to the separating gel. To dissolve lichenan (Sigma), the polysaccharide was mixed with water at room temperature to dissolve low molecular weight sugars; these were discarded. The remaining sugar was solubilised in water by heating at 80° C. for 30 min. CM cellulose (Sigma) also required heating at 80° C. for 30 min for solubilisation. 1.5 μg of thioredoxin. Pdlp2 and thioredoxin alone were loaded onto the gel and electrophoresed at 120 mV for 3 hours at room temperature. BSA (2 μg) was run as an additional negative control.

The proteins where visualised using Coomassie blue staining.

### Example 18

### Bioinformatics

Homologues of the Pdlp proteins were identified using the SMART database, (http://smart.embl-heidelberg.de) and BLAST. The homologues were aligned using Clustalw (http://www.ebi.ac.uk/clustalw/) and the alignment from Clustalw was put into the Tree top Phylogenetic tree prediction (http://www.genebee.msu.su/services/phtree_reduced.html) using the Phylip format. The distances relationships from this prediction were used to construct the a phylogenetic tree using Phylodendron tree printer (http://iubio.bio.indiana.edu/treeapp/treeprint-sample1.html).

### Example 19

### Cell-to-Cell Trafficking Assay

GFP is known to diffuse through plasmodesmata from one cell to the next. This is exploited by bombarding a CaMV 35S promoter driven expression plasmid into leaf tissues. The bombarded cell gives a high level of GFP expression and some of this GFP then diffuses into adjacent cells. By counting the number of surface cells making up the zone of diffusion, a quantitative measure of the diffusion is obtained. The interpretation is that smaller zones of diffusion reflect less active movement through plasmodesmata. Since GFP is not a plant protein and probably has no interacting partners in plants this diffusion is passive and a good measure of the physical properties of plasmodesmata. Sometimes the stochastic nature of the bombardment process means that bombardment of adjacent cells can confuse the data. To account for this we co-coat particles with the GFP expression plasmid and a plasmid expressing ER-targeted RFP, which will not move.

### Pdlp1 Overexpression

Overexpression of Pdlp1.GFP fusion in Arabidopsis resulted in plants with a dwarfed phenotype and an apparent reduction in traffiking though the plasmodesmata.

### Pdlp2 Overexpression

Overexpression of Pdlp2.GFP gave normal looking plants with an apparent increased accumulation of callose associated with the sites of overaccumulation of the protein. These sites were at plasmodesmata but also elsewhere outside of the cell wall. GFP trafficking, in these overexpression plants was indicated preliminarily to be reduced. Without wishing to be bound by mechanism, it therefore appears that Pdlp2 expression may be used to directly drive and hence modify callose deposition.

### REFERENCES AND NOTES

1. T. Kurata, K. Okada, T. Wada, Curr. Opin. Plant Biol. 8, 1 (2005).
2. J-Y. Lee et al., Proc. Natl. Acad. Sci. USA 103, 6055 (2006).
3. P. Dunoyer, C. Himber, O. Voinnet, Nat. Genet. 37, 1356 (2005).
4. W. J. Lucas, Virology 344, 169 (2006).
5. K. J. Oparka, Trend. Plant Sci. 9, 33 (2004).
6. T. Gillespie et al., Plant Cell. 14, 1207 (2002)
7. S. von Bargen, K. Salchert, M. Paape, B. Piechulla, J. W. Kellmann Plant Physiol. Biochem. 39, 1083 (2001).
8. V. Boyko, J. Ferralli, M. Heinlein, Plant J 22, 315 (2000)
9. A. I. Prokhnevsky, V. V. Peremyslov, V. V. Doija J. Virol. 79, 14421 (2005)
10. F. Kragler, M. Curin, K. Trutnyeva, A. Gansch, E. Waigmann Plant Physiol. 132, 1870 (2003).
11. C. Laporte, et al., Plant Cell 15, 2058 (2003)
12. E. Bayer, C. L. Thomas, A. J. Maule Protoplasma 223, 93 (2004)
13. J. E. Radford, M. Vesk, R. L. Overall Protoplasma 201, 30 (1998)
14. B. A. Stone and A. E. Clarke Chemistry and biology of (1-3) beta glucans. La Trobe University Press, Australia (1992)
15. J. E. Hughes, B. E. S. Gunning Can. J. Bot. 58, 250 (1980)
16. Y. L. Ruan, S. M. Xu, R. White, R. T. Furbank Plant Physiol. 136, 4104 (2004)
17. P. L. H. Rinne, P. M. Kaikuranta, C. Van der Schoot Plant J. 26, 249 (2001)
18. V. A. Iglesias, F. Meins Plant J. 21,157 (2000)
19. C. Bucher et al., Plant J. 28, 361 (2001)
20. P. L. H. Rinne et al., Plant J. 43, 688 (2005)
21. M. Sivaguru, et al., Plant Physiol. 124, 991 (2000)
22. P. Barral, et al., Biochemical J. 390, 77 (2005)
23. G. Sagi, A. Katz, D. Guenoune-Gelbart, B. Epel Plant Cell 17, 1788 (2005)
24. Z. Chen Plant Physiol 126, 473 (2001)
25. P. Czernic et al., Plant J. 18, 321 (1999).
26. L. Du, Z. Chen Plant J. 24, 837 (2000).
27. Y. Ohtake, T. Takahashi, Y. Komeda Plant Cell. Physiol. 41, 1038 (2000).
28. B. Henrissat, P. M. Coutinho, G. J. Davies Plant Mol. Biol. 47, 55 (2001)
29. M. Karimi, B. De Meyer, P. Hilson Trends Plant Sci. 10,103 (2005)
30. K. Earley, et al., Plant J. 45, 616 (2006).
31. S. J. Clough, A. F. Bent Plant J. 16, 735 (1998).
32. E. M. Bayer, et al., Proteomics 6, 301 (2006).
33. https://www.genevestigator.ethz.ch/
34. F. Brandizzi et al., Plant Cell 14, 1077 (2002)

Sequence Annex

DEFINITION Arabidopsis thaliana unknown protein (AT5G43980)
mRNA, complete cds.

| FEATURES | Location/Qualifiers |
|---|---|
| source | 1 . . . 1213 |
| | /organism = “Arabidopsis thaliana” |
| | /mol_type = “mRNA” |
| | /db_xref = “taxon: 3702” |
| | /chromosome = “5” |
| | /ecotype = “Columbia” |
| gene | 1 . . . 1213 |
| | /locus_tag = “AT5G43980” |
| | /note = “synonyms: MRH10.9, MRH10_9” |
| | /db_xref = “GeneID: 834421” |
| CDS | 165 . . . 1076 |
| | /locus_tag = “AT5G43980” |
| | /go_component = “endomembrane system” |
| | /go_function = “molecular function unknown” |
| | /go_process = “biological process unknown” |
| | /note = “receptor-like protein kinase-related, similar to |
| | receptor-like protein kinase homolog RK20-1 (GI: 4530126) |
| | (Phaseolus vulgaris); contains Pfam PF01657: Domain of |
| | unknown function” |
| | /codon_start = 1 |
| | /product = “unknown protein” |
| | /protein_id = “NP 199211.2” |
| | /db_xref = “GI: 30694417” |
| | /db_xref = “GeneID: 834421” |

/translation = “MKLTYQFFIFWFFLPFFAISGDDDYKNLIFKGCANQKSPDPTGV
FSQNLKNLFTSLVSQSSQSSFASVTSGTDNTTAVIGVFQCRGDLQNAQCYDCVSKIPK
LVSKLCGGGRDDGNVVAARVHLAGCYIRYESSGFRQTSGTEMLFRVCGKKDSNDPGFV
GKRETAFGMAENGVKTGSSGGGGGGGGFYAGQYESVYVLGQCEGSLGNSDCGECVKDG
FEKAKSECGESNSGQVYLQKCFVSYSYYSHGVPNIEPLSGGEKRQHTERTIALAVGGV
FVLGFVIVCLLVLRSAMKKKSNKYDAY”

ORIGIN

| | |
|---|---|
| 1 | acattttttt gtctttaaac ctcaaaacaa aagacaaaaa aaaaacggaa aactttcttt |
| 61 | tttccggaca cataaaaaca acacaaactt tattagacag aaacaaacta gtaaaaagat |
| 121 | tttgaacatc aagaaacaaa aatctctctc tgtctttctc aattatgaaa ctcacctatc |
| 181 | aattcttcat cttctggttc tttttaccat tctttgcaat ctccggtgat gatgattata |
| 241 | aaaatctgat ctttaaaggt tgtgcaaatc agaaatctcc agacccaact ggtgttttct |
| 301 | ctcagaatct caaaaactta ttcacttctt tagtttctca atcatcacaa agctctttcg |
| 361 | cttccgtaac ctccggaact gataacacca ccgccgtgat cggtgttttt cagtgccgtg |
| 421 | gcgatctcca aaacgctcag tgttacgatt gcgtctccaa aatccctaaa ctcgtttcta |
| 481 | aactctgcgg tggcggcaga gatgacggta atgtggtggc ggctcgtgtt cacctcgctg |
| 541 | gatgttatat ccggtatgag agttcaggat tccggcaaac ttccggtacg gagatgttgt |
| 601 | tccgtgtctg tgggaaaaaa gattctaacg atcctgggtt tgtcgggaaa agggagacgg |
| 661 | cgtttggtat ggcggagaac ggcgtgaaaa ctggatcatc cggtggtggt ggtggcggag |
| 721 | gaggttttta cgcggggcag tatgagtcgg tgtatgtgtt gggacagtgt gagggtagtt |
| 781 | tgggaaattc agattgtggg gaatgtgtga aagatggatt tgagaaagca aagagtgagt |
| 841 | gtggagagtc gaactcggga caagtttacc ttcagaagtg cttcgttagc tatagttact |
| 901 | actctcatgg tgttcccaac atagagccat tatcaggtgg agagaagaga caacacacag |
| 961 | aaaggacgat agctttggcg gtgggaggag tttttgtttt agggtttgtg attgtttgtt |
| 1021 | tgttggtttt gaggtctgcc atgaagaaga agagtaataa atatgatgct tattgattct |
| 1081 | tatttttacc taccaattct tattttccca attaaaaatt taacaaggta tattagaaaa |
| 1141 | atgattagta tagcatgtcc aattgtatat tcaattatga tattgattta aatacaaata |
| 1201 | aattttgatt ttt |

DEFINITION Arabidopsis thaliana unknown protein (AT5G61130)
mRNA, complete cds.

| FEATURES | Location/Qualifiers |
|---|---|
| source | 1 . . . 1025 |
| | /organism = “Arabidopsis thaliana” |
| | /mol_type = “mRNA” |
| | /db_xref = “taxon: 3702” |
| | /chromosome = “5” |
| | /ecotype = “Columbia” |
| gene | 1 . . . 1025 |
| | /locus_tag = “AT5G61130” |
| | /note = “synonyms: MAF19.13, MAF19_13” |
| | /db_xref = “GeneID: 836234” |
| CDS | 212 . . . 817 |
| | /locus_tag = “AT5G61130” |
| | /go_component = “anchored to membrane [pmid 12068095] |
| | [pmid 12805588]” |
| | /go_function = “molecular function unknown” |
| | /go_process = “biological process unknown” |
| | /note = “glycosyl hydrolase family protein 17, similar to |
| | beta-1,3-glucanase GI: 15150341 from (Camellia sinensis); |
| | C-terminal homology only” |
| | /codon_start = 1 |
| | /product = “unknown protein” |
| | /protein_id = “NP 200921.2” |
| | /db_xref = “GI: 30697478” |
| | /db_xref = “GeneID: 836234” |

/translation = “MAALVLSLLLLSLAGHSSASWCVCKTGLSDTVLQATLDYACGNG
ADCNPTKPKQSCFNPDNVRSHCNYAVNSFFQKKGQSPGSCNFDGTATPTNSDPSYTGC
AFPTSASGSSGSTTVTPGTTNPKGSPTTTTLPGSGTNSPYSGNPTNGVFGGNSTGGTT
GTGINPDYTTDSSAFALKNSSKLFICLLLIASSGFCSFLML”

ORIGIN

| | |
|---|---|
| 1 | gagaagagac aagaagacac agaacaagta gccactctct catctcacca cccctctttc |
| 61 | tctctacttg ctctctatct ctctctgtgt ctctccactt tctcttcttc ttcttcttct |
| 121 | tcttcttctt gctctagaga tctctctatc tccattcttt tctctctctg tttctagaaa |
| 181 | gacaacagtg acattttgtc tgaacgcatc tatggctgct ctggtgcttt cacttctcct |
| 241 | tctatccttg gctggacatt ctagtgcctc atggtgtgtg tgtaagacag ggctgagtga |
| 301 | tacagtgcta caggcaaccc tagactatgc ttgtggaaat ggagcagatt gtaatcctac |
| 361 | taaaccaaaa caatcttgct tcaaccctga caatgttagg tctcattgca actatgcagt |
| 421 | caatagcttc ttccaaaaga agggtcaatc tcctggctct tgtaatttcg atggaactgc |
| 481 | cactcccact aactccgatc ccagttatac aggttgtgcc tttcctacta gtgccagtgg |
| 541 | ctctagcggc agcacaactg tgacacctgg cacaaccaat ccaaaaggca gcccaacgac |
| 601 | caccacactt cctggtagtg gtaccaacag tccttattca gggaacccaa ccaatggagt |
| 661 | ttttggggga aatagcacag gaggcaccac tgggacaggg attaacccgg attacacgac |
| 721 | agacagcagc gcgtttgctc tcaagaactc aagcaaattg ttcatctgcc ttctcttgat |
| 781 | cgcttcgagt ggattctgct ctttcctgat gctctaagga tgttaatggt ctctggttct |
| 841 | gtgggtgcac ttagttatcg tttcgaggac attttggtct agtttagtgg taatctctgt |
| 901 | ctctgtgtta tgggtctatg ttatgttttt gtttgtaaga agatttcagc tcttgcagtt |
| 961 | actcttaatt acctctttga ctgttctggt agatttgatg ccagtgaaaa aaaatcttaa |
| 1021 | tatat |

1. An isolated polypeptide which is
(i) a plasmodesmata-associated protein, or
(ii) an isolated component of a plasmodesmata-associated protein,
which polypeptide is sufficient to target a protein linked thereto to the plasmodesmal space.

2. The polypeptide of claim 1 wherein the plasmodesmata-associated protein is selected from the group consisting of Pdlp1 and Pdlp2 and equivalents thereof.

3. The polypeptide of claim 2 wherein the equivalent shares at least 70% sequence identity with Pdlp1 or Pdlp2

4. The polypeptide of claim 2, which is an isolated component of a plasmodesmata-associated protein, which component comprises or consists of a transmembrane domain (TMD) which is a consensus TMD of Pdlp1 and equivalents thereof.

5. The polypeptide of claim 4 which is an isolated component of Pdlp1 comprising or consisting of the transmembrane domain (TMD) of Pdlp1.

6. The polypeptide of claim 1, which is an isolated component of Pdlp2.

7. The polypeptide of claim 2, which is an isolated component of a plasmodesmata-associated protein, which component comprises or consists of a Glycosyl phophotidylnositol-anchor protein specifying targeting to the extracellular domain of the plasma membrane for interaction with the cell wall.

8. A fusion polypeptide comprising the polypeptide of claim 1 fused to a protein of interest.

9. A method for targeting a protein of interest to the plasmodesmal milieu, which method comprises linking said protein of interest to a polypeptide defined in claim 1 which is sufficient to target said protein of interest linked thereto to the exterior or interior of the plasmodesmal space.

10. The method according to claim 9 for controlling flux through plant plasmodesmata, which comprises modifying the composition of the plasmodesmata by targeting to the neck of the plasmodesmata a protein of interest that modifies the aperture of the flux channel either directly or indirectly by modification of the effectiveness of callose deposition.

11. The method according to claim 9 for controlling flux through plant plasmodesmata, which comprises modifying the composition of the plasmodesmata by targeting within the plasmodesmata a protein of interest that modifies said flux.

12. The method according to claim 11 wherein the protein of interest is modifiable or cleavable in response to a stimulus.

13. The method according to claim 9 wherein the protein of interest has a defined binding specificity to plasmodesmata such as to modify specific molecular flux through plasmodesmata.

14. The method according to claim 13 wherein the protein of interest is an enzyme or antibody.

15. A method for controlling flux through plant plasmodesmata, which method comprises modifying naturally or artificially the expression levels or location of Pdlp1 or Pdlp2 or a homolog or ortholog thereof.

16. A process for producing a polypeptide of claim 1, which process comprises recombinantly expressing the polypeptide from heterologous nucleic acid encoding therefor, in a plant cell.

17. A method as claimed in claim 9 comprising transforming a plant cell or an ancestor thereof with heterologous nucleic acid encoding the protein of interest and polypeptide such as to express them as a fusion protein.

18. A recombinant plant vector which comprises a nucleic acid encoding the polypeptide of claim 1.

19. A process for producing a transgenic plant, which method comprises the steps of:
(a) transforming a plant cell with a heterologous nucleic acid encoding a polypeptide which is a plasmodesmata-associated protein, or an isolated component of a plasmodesmata-associated protein, which polypeptide is sufficient to target a protein linked thereto to the plasmodesmal space; and
(b) regenerating a plant from the transformed plant cell.

20. A transgenic plant comprising a heterologous nucleic acid encoding a polypeptide which is a plasmodesmata-associated protein, or an isolated component of a plasmodesmata-associated protein, which polypeptide is sufficient to target a protein linked thereto to the plasmodesmal space.

21. The transgenic plant of claim 20, wherein the plant was obtained by:
(a) transforming a plant cell with a heterologous nucleic acid encoding the polypeptide; and
(b) regenerating the plant from the transformed plant cell.

22. The transgenic plant of claim 20, wherein the plant is a clone, or selfed or hybrid progeny or other descendant of a plant regenerated from a transformed plant cell comprising a heterologous nucleic acid encoding the polypeptide.
